(19) United States
(12) Patent Application Publication
(10) Pub. No.: US 2024/0170156 A1
(43) Pub. Date: May 23, 2024

(54) RISK INDEX SYSTEM FOR EVALUATING RISK OF DIABETIC RETINOPATHY

(71) Applicant: The Board of Regents for the Oklahoma Agricultural and Mechanical Colleges, Stillwater, OK (US)

(72) Inventors: Ru Wang, Stillwater, OK (US); Zhuqi Miao, Stillwater, OK (US); Tieming Liu, Stillwater, OK (US); William Paiva, Tulsa, OK (US); Dursun Delen, Tulsa, OK (US)

(21) Appl. No.: 18/425,846

(22) Filed: Jan. 29, 2024

Related U.S. Application Data
(63) Continuation of application No. PCT/US2022/074258, filed on Jul. 28, 2022.
(60) Provisional application No. 63/227,150, filed on Jul. 29, 2021.

(51) Int. Cl. G16H 50/30; G16H 10/60

(52) U.S. Cl. CPC G16H 50/30; G16H 10/60

(57) ABSTRACT

A non-transitory computer readable medium is described. The non-transitory computer readable medium has computer executable instructions that when executed cause a processor to: determine a diabetic retinopathy prediction based at least in part on a weighted sum of at least five patient predictors in a database storing at least one patient record having first data; and pass an alert to a user responsive to the diabetic retinopathy prediction being within a predetermined range.

### CROSS-REFERENCE TO RELATED APPLICATIONS/INCORPORATION BY REFERENCE STATEMENT

The present patent application is a continuation of International Application No. PCT/US2022/074258, filed Jul. 28, 2022; which claims priority to the provisional patent application identified by U.S. Ser. No. 63/227,150, filed on Jul. 29, 2021. The entire content of International Application No. PCT/US2022/074258 and U.S. Ser. No. 63/227,150 are hereby incorporated by reference herein.

### STATEMENT REGARDING FEDERALLY SPONSORED RESEARCH OR DEVELOPMENT

Not applicable.

### BACKGROUND

Diabetic retinopathy (also referred to herein as “DR”) is a vision-threatening microvascular complication of diabetes and is a leading cause of blindness among working-aged adults globally. According to the 2002 American Diabetes Association Position Statement, nearly 100% of patients with type 1 diabetes and over 60% of patients with type 2 diabetes developed retinopathy during the first 20 years of the disease. In 2015, about 1.5 million Americans were diagnosed with diabetes, and an additional 84.1 million Americans had prediabetes. The rapidly growing number of new diabetes patients suggests that DR will continue to be a major cause of vision loss and associated functional impairment in the U.S. in the coming years.

Because DR can progress to irreversible stages (i.e., at which point it is impossible to restore visual acuity) with relatively few symptoms, early detection and treatment are critical in preventing DR and the subsequent vision loss. Although DR diagnostic and treatment options have significantly advanced over the past decades, early detection and screening for DR remain challenging due to poor adherence to annual examination guidelines and a lack of resources to deploy comprehensive screening programs, especially in rural or undeveloped areas. Therefore, there is a desire to research innovative ways to implement timely, cost-effective detection techniques and/or programs.

Clinical predictive models provide an effective alternative solution to improve the access to early screening for DR under current limitations, by forecasting accurate risk estimates of diseases based on important biomarkers. Predictive models have been extensively investigated and adopted in diabetes studies. In particular for DR, many conditions comorbid with diabetes, such as hyperglycemia, hypertension and dyslipidemia have been found to be associated with DR. In addition, HbA1c, fasting plasma glucose, hemoglobin, hematocrit, and many other laboratory test values were found to be risk factors for DR development. Based on the risk factors identified, a few prediction models were developed to predict the incidence and development of DR. However, most of the prediction models incorporated a multitude of laboratory variables, leading to no consensus about which laboratory tests are required for effective and economical prediction of DR.

Accordingly, there is a need for an accurate and cost-effective predictive model and an easy-to-use risk index for assisting healthcare providers in identifying patients at high DR risk and counseling them for ophthalmic examination and proper treatments at early stages.

### SUMMARY

DR is a major cause of blindness among middle-aged adults over the world. Vision loss which occurs at the late stage of DR cannot be reversed. As a result, diagnosing DR at an early date is very desirable. The present disclosure describes a system for calculating the risk of developing DR for a patient with diabetes and a method of using the same in a practical application. The system and method may apply a risk index to a number of patient predictors for DR, which may include the patient's age, status on neuropathy and nephropathy, and results for analyzing multiple analytes, e.g., eight analytes (i.e., creatinine, HbA1c, white blood cell, glucose, hematocrit, anion gap, potassium, and sodium) along with other patient data. Using the patient predictors as inputs, a user may employ the presently described system and method to evaluate the patient's risk of developing DR and perform an action such as alerting the user to see an ophthalmologist for eye examination and potentially follow-up actions recommended by the ophthalmologist.

In one embodiment, the present disclosure describes a non-transitory computer readable medium having computer executable instructions that when executed cause a processor to: determine a diabetic retinopathy prediction based at least in part on a weighted sum of at least five patient predictors in a database storing at least one patient record having first data; and pass an alert to a user responsive to the diabetic retinopathy prediction being within a predetermined range.

One technological improvement of the presently described system, non-transitory computer readable medium and method is the determination of a subset of patient predictors 18 that contribute a majority of predictive accuracy and development of the first algorithm that improves the functioning of the processor by reducing clock cycles as compared to existing methodologies using machine learning techniques. As a result, data collection may be less expensive and collected data may be easier to interpret. Additionally, while existing methods for predicting DR utilize machine learning and may achieve high predictive accuracy, the machine learning algorithms require significant processing power and memory, and the “black box” nature of such methods make them difficult for a user to understand. Moreover, complex machine learning algorithms require the support of specific software (e.g., R) for their execution, which may be less user-friendly and may increase the cost of use. In order to address these concerns, another improvement of the presently described system and method is the development of a risk index and the optional inclusion of a graphical user interface. The DR prediction given in accordance with the present disclosure may be used as an early warning sign to urge patients to undergo an ophthalmic examination, which has a relatively low compliance rate currently.

### BRIEF DESCRIPTION OF THE DRAWINGS

FIG. 1 is a block diagram of an exemplary embodiment of a DR risk evaluation system constructed in accordance with the present disclosure.

FIG. 2 is a process flow diagram of a method for evaluating a risk of DR in accordance with the present disclosure.

FIG. 3 is a timeline for determining a DR prediction in accordance with the present disclosure.

FIG. 4A is a table depicting the prediction scores assigned to each of the patient predictors by the DR risk evaluation system of FIG. 1.

FIG. 4B is another embodiment of the table depicting the prediction scores assigned to each of the patient predictors by the DR risk evaluation system of FIG. 1.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

Circuitry, as used herein, may be analog and/or digital components, or one or more suitably programmed processors (e.g., microprocessors) and associated hardware and software, or hardwired logic. Also, “components” may perform one or more functions. The term “component,” may include hardware, such as a processor (e.g., microprocessor), an application specific integrated circuit (ASIC), field programmable gate array (FPGA), a combination of hardware and software, and/or the like. The term “processor” as used herein means a single processor or multiple processors working independently or together to collectively perform a task.

Software may include one or more computer readable instructions that when executed by one or more components cause the component to perform a specified function. It should be understood that the algorithms described herein may be stored on one or more non-transitory memory. Exemplary non-transitory memory may include random access memory, read only memory, flash memory, and/or the like. Such non-transitory memory may be electrically based, optically based, and/or the like.

The term “healthcare provider” as used herein includes a person or group of persons capable of providing health services including, but not limited to, a Doctor of Medicine or osteopathy, podiatrist, dentist, chiropractor, clinical psychologist, optometrist, nurse practitioner, nurse-midwife, nurse, a clinical social worker, veterinarian, and the like. Further, “healthcare provider” may include any provider whom an insurance provider will accept medical codes to substantiate a claim for benefits.

As used herein, the terms “network-based”, “cloud-based”, and any variations thereof, may include the provision of configurable computational resources on demand via interfacing with a computer and/or computer network, with software and/or data at least partially located on a computer and/or computer network, by pooling processing power of two or more networked processors.

The term “patient” as used herein includes human and veterinary subjects.

Referring now to the Figures, and in particular to FIG. 1, a DR risk evaluation system 10 may be one or more system that is able to embody and/or execute the logic of the processes described herein. Logic embodied in the form of software instructions and/or firmware may be executed on any appropriate hardware. For example, logic embodied in the form of software instructions or firmware may be executed on a system or systems, or on a personal computer system, or on a distributed processing computer system, and/or the like. In some embodiments, logic may be implemented in a stand-alone environment operating on a single computer system and/or logic may be implemented in a networked environment, such as a distributed system using multiple computers and/or processors networked together.

As shown in FIG. 1, certain non-limiting embodiments of the present disclosure may include the DR risk evaluation system 10 having a user device 14 operable to receive at least five patient predictors 18 (hereinafter “patient predictors 18”) from a user. The user may be, for example, a patient, a physician, and/or a medical technician. The patient predictors 18 may comprise one or more demographic status and/or one or more blood analyte.

The user device 14 may be provided with one or more processor 26 (hereinafter “processors 26”), one or more non-transitory computer readable medium 30 (hereinafter “memories 30”), and a communication device 34, the communication device 34 operable to communicate, via a network 38, with, for example, one or more external systems 42 (hereinafter “external systems 42”) (e.g., external computer systems, machine learning applications, artificial intelligence-based systems, cloud-based systems, etc.).

The user device 14 may be configured to provide information and/or data in a form perceivable to one or more processors 46 of the external systems 42. For example, the user device 14 may include, but is not limited to, implementations as a laptop computer, a desktop computer, a computer monitor, a screen, a touchscreen, a microphone, a website, a smart phone, a PDA, a cell phone, an optical head-mounted display, combinations thereof, and/or the like. The user device 14 may provide data (e.g., the patient predictors 18) in computer readable form, such as a text file, a word document, and/or the like.

The external systems 42 may be provided with one or more processors 46 (hereinafter “processors 46”), one or more non-transitory computer readable medium 50 (hereinafter “memories 50”), and a communication device 54, the communication device 54 operable to communicate via the network 38 with, for example, the user device 14.

The external systems 42 may be configured to provide information and/or data in a form perceivable to the processors 26 of the user device 14. For example, the external systems 42 may include, but are not limited to, implementations as a laptop computer, a desktop computer, a computer server, a computer monitor, a screen, a touchscreen, a microphone, a website, a smart phone, a PDA, a cell phone, an optical head-mounted display, combinations thereof, and/or the like. The external systems 42 may provide data (e.g., an eye examination recommendation alert, an eye examination frequency alert, and/or a readmission prediction alert) in computer readable form, such as a text file, a word document, and/or the like.

Exemplary embodiments of processors 26 and 46 may include, but are not limited to, a digital signal processor (DSP), a central processing unit (CPU), a field programmable gate array (FPGA), a microprocessor, a multi-core processor, and/or combinations thereof, for example. In some embodiments, the processors 26 and 46 may be incorporated into a smart device. It is to be understood, that in certain embodiments, using more than one processor, the processors 26 and 46 may be located remotely from one another, in the same location, or comprise a unitary multi-core processor. In some embodiments, the processors 26 and 46 may be partially or completely network-based or cloud-based, and may or may not be located in a single physical location. The processors 26 and 46 may be capable of reading and/or executing processor executable code and/or capable of creating, manipulating, retrieving, altering, and/or storing data structure into the respective memories 30 and 50.

In some embodiments, the processors 26 and 46 may transmit and/or receive data via the network 38. For example, the processors 26 may allow the user (e.g., a patient, a physician, medical personnel, etc.) to use the user device 14 to access the external systems 42 via the network 38 to provide and/or receive data, such as the patient predictors 18 and/or one or more patient record 62 (hereinafter “patient records 62”) (discussed in greater detail below). Access methods include, but are not limited to, cloud access and direct download to the user device 14 via the network 38. In some embodiments, the processors 26 and 46 may be provided on a cloud cluster (i.e., a group of nodes hosted on virtual machines and connected within a virtual private cloud). Additionally, the processors 26 and 46 may provide data to the user by methods that include, but are not limited to, messages sent through the user device 14 and/or the external systems 42, SMS, email, and telephone, to provide data such as an alert (e.g., an eye examination recommendation alert, an eye examination frequency alert, and/or a readmission prediction alert), for example. It is to be understood that in some exemplary embodiments, the user device 14 and the external systems 42 may be implemented as a single device.

The external systems 42 may be configured to provide information and/or data in a form perceivable to the processors 26. For example, the external systems 42 may include, but are not limited to, implementations as a laptop computer, a computer monitor, a screen, a touchscreen, a microphone, a website, a smart phone, a PDA, a cell phone, an optical head-mounted display, combinations thereof, and/or the like. The external systems 42 may provide data (e.g., an eye examination recommendation alert, an eye examination frequency alert, and/or a readmission prediction alert) in computer readable form, such as a text file, a word document, and/or the like.

The network 38 may be almost any type of network. For example, the network 38 may interface via optical and/or electronic interfaces, and/or may use a plurality of network topographies and/or protocols including, but not limited to, Ethernet, TCP/IP, circuit switched paths, combinations thereof, and the like. For example, in some embodiments, the network 38 may be implemented as the World Wide Web (or Internet), a local area network (LAN), a wide area network (WAN), a metropolitan network, a wireless network, a cellular network, a Global System of Mobile Communications (GSM) network, a code division multiple access (CDMA) network, a 4G network, a 5G network, a satellite network, a radio network, an optical network, an Ethernet network, combinations thereof, and/or the like. Additionally, the network 38 may use a variety of network protocols to permit bi-directional interface and/or communication of data and/or information. It is conceivable that in the near future, embodiments of the present disclosure may use more advanced networking topologies.

In some embodiments where the network 38 is the Internet, a primary user interface of the DR risk evaluation system 10 may be delivered through a series of web pages. It should be noted that the primary user interface of the DR risk evaluation system 10 may be via any type of interface, such as, for example, a Windows-based application.

At least one of the memories 30 and 50 may store processor executable code comprising one or more databases 56 (hereinafter “databases 56”) and/or program logic 58 (i.e., computer executable logic). In some embodiments, the processor executable code may be stored as a data structure, such as a database and/or data table, for example. The program logic 58 may comprise processor executable code that when executed may cause the processor 26 to evaluate a risk of diabetic retinopathy (discussed in greater detail below). In use, the processors 26 and 46 may execute the program logic 58 controlling the reading, manipulation, and/or storing of data as detailed in the processes described herein.

In some embodiments, the memories 30 and 50 may be located in the same physical location as the respective processors 26 and 46. Alternatively, the memories 30 and 50 may be located in a different physical location as the respective processors 26 and 46 and communicate with the respective processors 26 and 46 via a network, such as the network 38. Additionally, the memories 30 and 50 may be implemented as “cloud memories” (i.e., one or more memories may be partially or completely based on or accessed using a network, such as network 38).

The databases 56 may be stored on at least one of the memories 30 and 50 and may comprise the patient predictors 18 inputted by the user and/or one or more patient record 62 (hereinafter “patient records 62”), at least one of the patient records 62 having first data 66 corresponding to a patient. At least one of the patient records 62 may also have second data 70 corresponding to the patient (discussed in greater detail below).

The user device 14 may receive the patient predictors 18 from the user using one or more input device 74 (hereinafter “input devices 74”). The input devices 74 may include, but is not limited to, implementation as a keyboard, touchscreen, mouse, trackball, microphone, fingerprint reader, infrared port, slide-out keyboard, flip-out keyboard, cell phone, PDA, video game controller, remote control, network interface, speech recognition, gesture recognition, combinations thereof, and/or the like.

The user device 14 may provide data (e.g., an eye examination recommendation alert, an eye examination frequency alert, and/or a readmission prediction alert) to the user using one or more output device 78 (hereinafter “output devices 78”). The output device 78 may be capable of outputting information in a form perceivable by the user, the external systems 42, and/or the processors 26 and 46. For example, the output devices 78 may include, but are not limited to, implementations as a computer monitor, a screen, a touchscreen, a speaker, a website, a television set, a smart phone, a PDA, a cell phone, a fax machine, a printer, a laptop computer, an optical head-mounted display (OHMD), combinations thereof, and/or the like. It is to be understood that in some exemplary embodiments, the input devices 74 and output device 78 may be implemented as a single device, such as, for example, a touchscreen or a tablet.

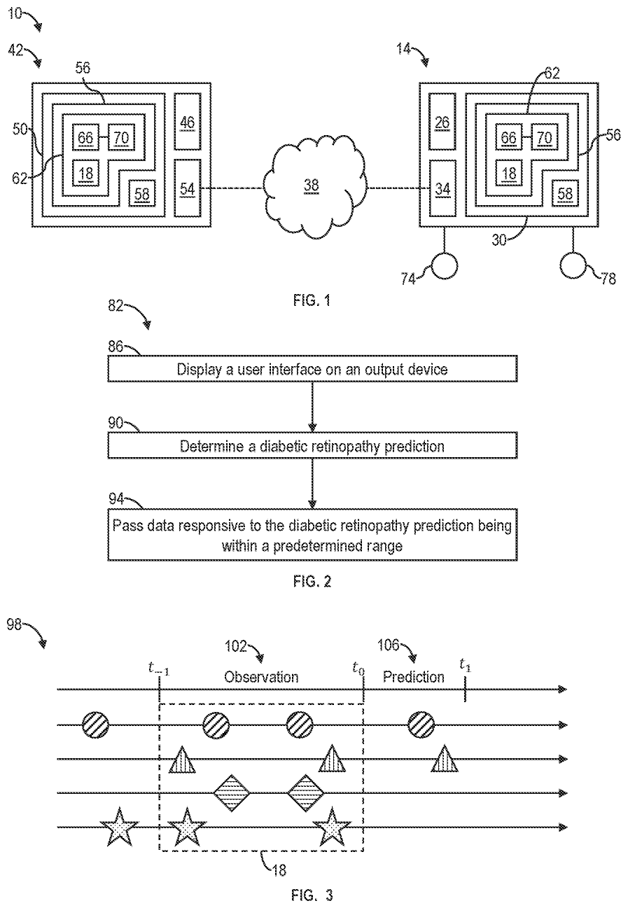

Referring now to FIG. 2, which in turn is a process flow diagram depicting a method 82 for evaluating a risk of DR. The method 82 may be performed by at least one of the processors 26 and 46 executing processor executable code (e.g., the program logic 58) and may comprise the steps of: displaying a user interface on the output device 78 (e.g., a display) (step 86); determining a DR prediction (step 90); and passing data responsive to the DR prediction being within a predetermined range (step 94).

The step of displaying the user interface on the output device 78 (e.g., a display) (step 86) may comprise displaying a user interface on the output device 78 (e.g., a display), wherein the user interface has one or more field (e.g., a text field) (hereinafter “fields”). Each of the fields may be operable to receive input from the user, which may include at least one of the patient predictors 18. In some embodiments, the fields are in a range from five fields to twenty fields, corresponding to an amount of the patient predictors 18. The user input (e.g., the patient predictors 18) may be stored in at least one of the patient records 62 in at least one of the databases 56.

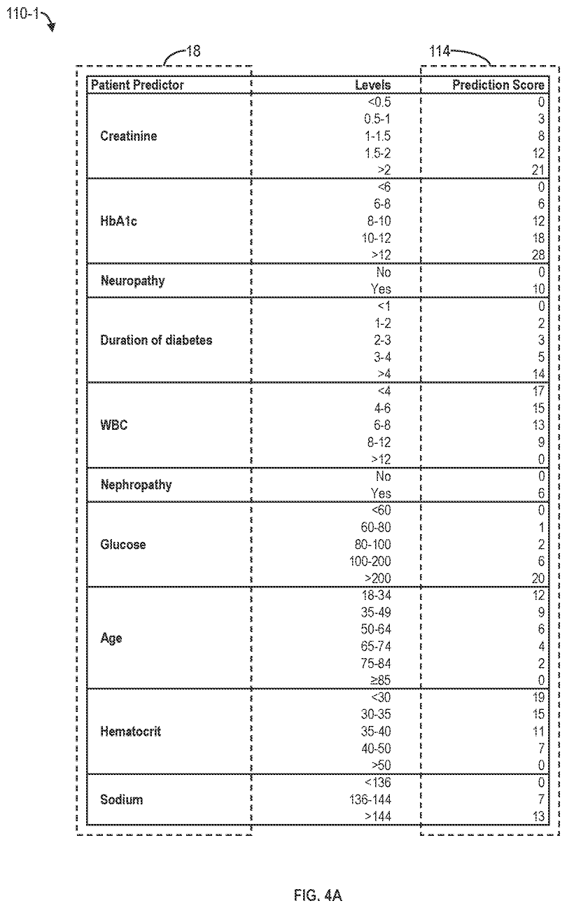

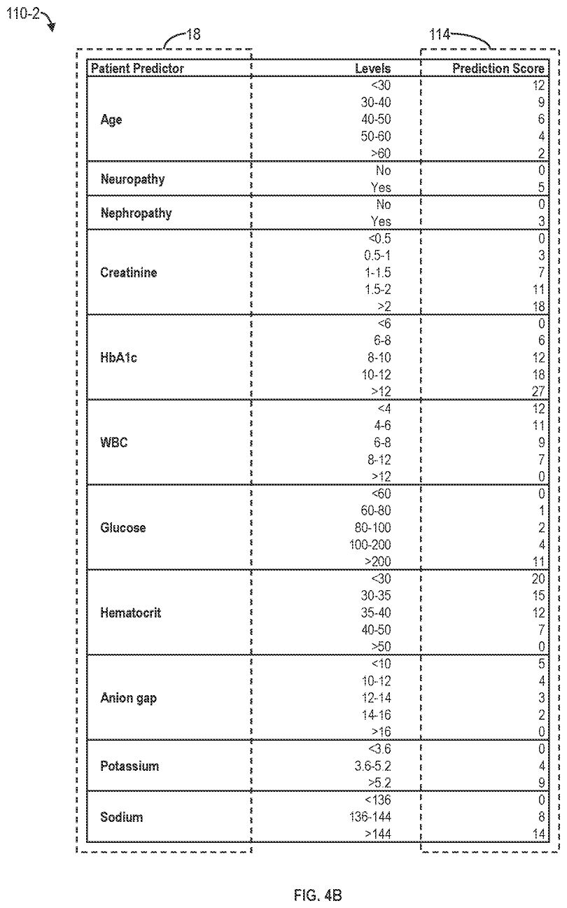

The step of determining the DR prediction (step 90) may comprise determining the DR prediction based at least in part on a weighted sum (see FIGS. 4A-4B) of one or more of the patient predictors 18, wherein the patient predictors 18 are stored in the databases 56, the databases 56 storing the patient records 62 comprising the first data 66. In some embodiments, the first data 66 may be indicative of an absence of a DR prediction. In some embodiments, the patient predictors 18 are at least five patient predictors. In some embodiments, the patient predictors 18 are in a range from five patient predictors 18 to ten patient predictors 18. In some embodiments where the patient predictors 18 have been previously stored in the databases 56, the method 82 may forgo the step of displaying the user interface on the output device 78 (e.g., a display) (step 86) and begin with the step of determining the DR prediction (step 90).

In some embodiments, the step of passing the data responsive to the DR prediction being within the predetermined range (step 94) may comprise passing an alert to the user responsive to the DR prediction being within the predetermined range. The alert may be provided in any form that is perceivable to the user (e.g., an aural form, a visual form, in the form of a text message, etc.). In some embodiments, the alert is indicative of an eye examination recommendation (i.e., an alert recommending that the user schedule an eye examination). In some embodiments, the alert is indicative of an eye examination frequency (i.e., an alert recommending that the user schedule a plurality of eye examinations at a particular frequency).

In some embodiments, the step of passing the data responsive to the DR prediction being within the predetermined range (step 94) may comprise updating the first data 66 of the patient records 62 with second data 70 responsive to the DR prediction being within the predetermined range. In some embodiments, the second data 70 may be indicative of a presence of a DR prediction.

In some embodiments, the step of determining the DR prediction (step 90) may be performed according to a first algorithm. The first algorithm may be operable to determine the DR prediction responsive to receiving the patient predictors 18. The first algorithm may be operable to assign a plurality of prediction scores 114 (see FIGS. 4A-4B), each of the prediction scores 114 being assigned to one of the patient predictors 18 (see FIGS. 4A-4B), and determine a weighted sum of the prediction scores 114, wherein a higher weighted sum is indicative of a greater risk of the patient developing DR. In some embodiments, the step of passing the data responsive to the DR prediction being within the predetermined range (step 94) may comprise passing data indicative of the DR prediction to a second algorithm responsive to the DR prediction being within the predetermined range. In some embodiments, the second algorithm may be operable to generate a readmission prediction (i.e., a likelihood that the user will be readmitted to hospital for DR-related symptoms). In some embodiments, the method 82 may further comprise the step of passing data (e.g., an alert) to the user responsive to the readmission prediction being within a predetermined range.

Referring now to FIG. 3, shown therein is a timeline 98 for determining a DR prediction in accordance with the present disclosure. In some embodiments, the DR risk evaluation system 10 and/or the method 82 for evaluating the risk of DR may be used and/or performed, respectively, according to the timeline 98. In some embodiments, the patient may be observed (i.e., one or more sample, e.g., a blood sample, may be obtained from the patient) during an observation window 102 (i.e., a time period between t₋₁ and t₀). The blood sample, for example, may include various analytes which can be measured by a medical analyzer. The measured analytes may be the patient predictors 18. The patient predictors 18 measured from the blood sample may be input into the user device 14 and analyzed by the first algorithm in order to determine the DR prediction at the beginning of a prediction window 106 (i.e., a time period between t₀ and t₁) using the patient predictors 18 (i.e., the one or more blood analytes measured during the observation window 102 and one or more demographic status of the patient predictors 18 observed at the beginning of the prediction window 106). The DR prediction may be indicative of a likelihood of the patient experiencing an onset of DR during the prediction window 106 (i.e., in the time period between t₀ and t₁). The observation window 102 may have a longer duration than the prediction window 106. In some embodiments, the prediction window 106 may be a period of six months before a potential diagnosis date (i.e., t₁) and the observation window 102 may be a period of two years immediately before the beginning of the prediction window 106 (i.e., t₀).

Referring now to FIG. 4A, shown therein is a table 110-1 depicting a plurality of prediction scores 114 assigned to each of the patient predictors 18 by the DR risk evaluation system 10 shown in FIG. 1. In some embodiments, the patient predictors 18 are at least five in number and may include, for example, creatinine, HbA1c, neuropathy, duration of diabetes (in months), and/or white blood cell (hereinafter “WBC”)). In some embodiments, the patient predictors 18 include one or more of nephropathy, glucose, age (in years), hematocrit, potassium, sodium, race, alanine aminotransferase (hereinafter “ALT”), hemoglobin, mean corpuscular hemoglobin concentration (hereinafter “MCHC”), red blood cell (hereinafter “RBC”), chlorine, calcium, albumin, aspartate aminotransferase (hereinafter “AST”), bilirubin, and mean corpuscular volume (hereinafter “MCV”).

For some of the patient predictors 18 indicative of an amount of a substance (e.g., an analyte) in the patient's blood (e.g., creatinine, HbA1c, glucose, potassium, and/or sodium), a higher prediction score 114 may be assigned where a greater amount of the substance is observed in the patient's blood. For others of the patient predictors 18 indicative of an amount of the substance in the patient's blood (e.g., WBC and hematocrit), a lower prediction score 114 may be assigned where a greater amount of the substance is observed in the patient's blood.

For some of the patient predictors 18 indicative of a length of time (e.g., duration of diabetes), a higher prediction score 114 may be assigned where the length of time is longer (e.g., the patient has been diagnosed with diabetes for a longer period). For others of the patient predictors 18 indicative of a length of time (e.g., age), a lower prediction score 114 may be assigned where the length of time is longer (e.g., the patient is older).

For the patient predictors 18 indicative of the existence or nonexistence of a complication (e.g., neuropathy and nephropathy), a nonzero prediction score 114 (e.g., a score of 3, 5, 6, 8, 10, etc.) may be assigned where the patient exhibits the complication and a zero prediction score 114 may be assigned where the patient does not exhibit the complication.

Returning to FIG. 2, in some embodiments, the step of determining the DR prediction (step 90) may comprise the steps of: assigning a prediction score for each of the patient predictors 18 according to levels of the patient predictors 18. Shown in FIG. 4A is a table 110-1 having various levels for each patient predictor 18, and prediction scores assigned to each of the levels. For example, when the patient predictor 18 is creatinine, five levels may be assigned. The levels can be <0.5; 0.5-1; 1-1.5; 1.5-2; and >2. As shown in the table of FIG. 4A, as the level of creatinine increases, the prediction score 114 for creatinine increases. Similarly, as the levels of HbA1C, duration of diabetes, glucose, and sodium increase, the prediction scores 114 also increase. The prediction scores 114, however, for some of the patient predictors 18 are inversely correlated with a patient's risk for DR. For example, as WBC, patient age, and hematocrit decrease, the prediction scores 114 increase indicating that the patient is at a higher risk of DR. The first algorithm may calculate a weighted sum of the prediction scores for each of the patient predictors 18, wherein the DR prediction is equal to the weighted sum. Further, in some embodiments, the step of passing the data responsive to the DR prediction being within the predetermined range (step 94) may comprise passing the data responsive to the DR prediction being within the predetermined range. In one example using the prediction scores 114 in FIGS. 4A and 4B, when the weighted sum is <=60, the risk is low with less than 10% patients developing diabetic retinopathy. When the weighted sum is between 60-80, e.g., 60<weighted sum <=80, the risk is medium, with no more than 25% patients developing diabetic retinopathy. When the weighted sum is greater than 80, the risk is high with more than 25% patients developing diabetic retinopathy.

Referring now to FIG. 4B, shown therein is another embodiment of a table 110-2 depicting a plurality of prediction scores 114 assigned to each of the patient predictors 18 by the DR risk evaluation system 10 shown in FIG. 1. As shown in FIG. 4B, in some embodiments, the patient predictors 18 may include, for example, anion gap. For the patient predictors 18 indicative of an acid-base balance of the patient's blood (e.g., anion gap), a higher prediction score 114 may be assigned where a greater amount of acidity is observed in the patient's blood.

### EXAMPLES

Examples are provided hereinbelow. However, the present disclosure is to be understood to not be limited in its application to the specific experimentation, results, and laboratory procedures disclosed herein. Rather, the Examples are simply provided as one of various embodiments and are meant to be exemplary, not exhaustive.

### Example 1

| | |
|---|---|
| Value | Score |

| | | | | |
|---|---|---|---|---|
| Patient | 1 | 2 | 1 | 2 |

Patient with high score and developed DR

| | | | | |
|---|---|---|---|---|
| HbA1C | 10.6 | 13.9 | 6 | 12 |
| Creatinine | 2.1 | 8.8 | 21 | 21 |
| Glucose | 290.1 | 234.4 | 18 | 28 |
| Hematocrit | 30.7 | 33.6 | 10 | 10 |
| Sodium | 137.1 | 138.6 | 14 | 5 |
| WBC | 3.8 | 10.6 | 17 | 9 |
| Age | 55.5 | 30.5 | 6 | 6 |
| Nephropathy | yes | yes | 20 | 20 |
| Neuropathy | yes | yes | 15 | 15 |
| Diabetic Duration | 4.6 | 3.6 | 7 | 7 |

| | | |
|---|---|---|
| Total | 134 | 133 |

Patient with low score and did not develop DR

| | | | | |
|---|---|---|---|---|
| HbA1C | 5.1 | 5.8 | 6 | 0 |
| Creatinine | 0.8 | 0.9 | 3 | 3 |
| Glucose | 95.4 | 81.0 | 0 | 0 |
| Hematocrit | 27.1 | 54.5 | 0 | 0 |
| Sodium | 133.7 | 139.1 | 2 | 0 |
| WBC | 13.2 | 16.5 | 0 | 0 |
| Age | 55.5 | 49.5 | 0 | 0 |
| Nephropathy | no | no | 2 | 2 |
| Neuropathy | no | no | 0 | 0 |
| Diabetic Duration | 1.2 | 0.6 | 0 | 7 |

| | | |
|---|---|---|
| Total | 13 | 12 |

In a non-limiting example, at the beginning of the prediction window 106, the DR risk evaluation system 10 may display the user interface on the output device 78, which may prompt a user, using the input device 74, to input the patient predictors 18 for a patient observed (i.e., one or more sample, e.g., a blood sample, was obtained from the patient) during the observation window 102, for example:

| Patient Predictor | Level |
|---|---|
| Creatinine | 1.7 |
| HbA1c | 8.1 |
| Neuropathy | No |
| Duration of diabetes | 2.5 |
| WBC | 9.6 |
| Nephropathy | Yes |
| Glucose | 84 |
| Age | 52 |
| Hematocrit | 32 |
| Sodium | 140 |

Responsive to the user inputting the patient predictors 18 for the patient, the DR risk evaluation system 10 may determine the DR prediction according to the first algorithm operable to assign the prediction scores 114 to each of the patient predictors 18 (as shown in FIG. 4A), for example:

| Patient Predictor | Level | Prediction Score |
|---|---|---|
| Creatinine | 1.7 | 12 |
| HbA1c | 8.1 | 12 |
| Neuropathy | No | 0 |
| Duration of diabetes | 2.5 | 3 |
| WBC | 9.6 | 9 |
| Nephropathy | Yes | 6 |
| Glucose | 84 | 2 |
| Age | 52 | 6 |
| Hematocrit | 32 | 15 |
| Sodium | 140 | 7 |

Subsequent to assigning the prediction scores 114 to each of the patient predictors 18, the DR risk evaluation system 10 may determine a weighted sum of the prediction scores 114, for example:

| Patient Predictor | Prediction Score |
|---|---|
| Creatinine | 12 |
| HbA1c | 12 |
| Neuropathy | 0 |
| Duration of diabetes | 3 |
| WBC | 9 |
| Nephropathy | 6 |
| Glucose | 2 |
| Age | 6 |
| Hematocrit | 15 |
| Sodium | 7 |

In this non-limiting example, the DR risk evaluation system 10 may determine the weighted sum (and therefore the DR prediction) to be 12+12+0+3+9+6+2+6+15+7=72, which may cause the DR risk evaluation system 10 to pass the data (e.g., an alert) responsive to the DR prediction being within the predetermined range.

### Example 2

In another non-limiting example, at the beginning of the prediction window 106, the DR risk evaluation system 10 may display the user interface on the output device 78, which may prompt a user, using the input device 74, to input the patient predictors 18 for a patient observed (i.e., one or more sample, e.g., a blood sample, was obtained from the patient) during the observation window 102, for example:

| Patient Predictor | Level |
|---|---|
| Creatinine | 0.4 |
| HbA1c | 6.1 |
| Neuropathy | Yes |
| Duration of diabetes | 0.5 |
| WBC | 5.5 |
| Nephropathy | No |
| Glucose | 62 |
| Age | 36 |
| Hematocrit | 28 |
| Sodium | 122 |

Responsive to the user inputting the patient predictors 18 for the patient, the DR risk evaluation system 10 may determine the DR prediction according to the first algorithm operable to assign the prediction scores 114 to each of the patient predictors 18 (as shown in FIG. 4A), for example:

| Patient Predictor | Level | Prediction Score |
|---|---|---|
| Creatinine | 0.4 | 0 |
| HbA1c | 6.1 | 6 |
| Neuropathy | Yes | 10 |
| Duration of diabetes | 0.5 | 0 |
| WBC | 5.5 | 15 |
| Nephropathy | No | 0 |
| Glucose | 62 | 1 |
| Age | 36 | 9 |
| Hematocrit | 28 | 19 |
| Sodium | 122 | 0 |

Subsequent to assigning the prediction scores 114 to each of the patient predictors 18, the DR risk evaluation system 10 may determine a weighted sum of the prediction scores 114, for example:

| Patient Predictor | Prediction Score |
|---|---|
| Creatinine | 0 |
| HbA1c | 6 |
| Neuropathy | 10 |
| Duration of diabetes | 0 |
| WBC | 15 |
| Nephropathy | 0 |
| Glucose | 1 |
| Age | 9 |
| Hematocrit | 19 |
| Sodium | 0 |

In this non-limiting example, the DR risk evaluation system 10 may determine the weighted sum (and therefore the DR prediction) to be 0+6+10+0+15+0+1+9+19+0=60. Because the DR prediction in this non-limiting example is not within the predetermined range, the DR risk evaluation system 10 may pass data (e.g., a notification) to the user including recommendations for reducing known modifiable risk factors for diabetes, such as recommendations involving weight control, smoking, alcohol use, exercise, blood sugar control, etc. to mitigate risks of DR. Other notifications for non-modifiable risk factors, such as genetics, age, etc. may also be included.

The following is a number list of non-limiting illustrative embodiments of the inventive concept disclosed herein:

1. A non-transitory computer readable medium having computer executable instructions that when executed cause a processor to:
determine a diabetic retinopathy prediction based at least in part on a weighted sum of at least five patient predictors in a database storing at least one patient record having first data; and
pass an alert to a user responsive to the diabetic retinopathy prediction being within a predetermined range.

2. The non-transitory computer readable medium of illustrative embodiment 1, wherein the user is at least one of a patient and a physician.

3. The non-transitory computer readable medium of any of illustrative embodiments 1 to 2, wherein the alert is provided in at least one of an aural form or a visual form.

4. The non-transitory computer readable medium of any of illustrative embodiments 1 to 2, wherein the alert is indicative of at least one of an eye examination recommendation and an eye examination frequency.

5. The non-transitory computer readable medium of any of illustrative embodiments 1 to 5, wherein the at least five patient predictors are in a range from five patient predictors to ten patient predictors.

6. The non-transitory computer readable medium of any of illustrative embodiments 1 to 5, further comprising computer executable instructions that when executed cause the processor to display a user interface on a display, the user interface having a plurality of fields operable to receive input from a user, the input indicative of the at least five patient predictors.

7. A non-transitory computer readable medium having computer executable instructions that when executed cause a processor to:
determine a diabetic retinopathy prediction based at least in part on a weighted sum of at least five patient predictors in a database storing at least one patient record having first data; and
update the first data with second data responsive to the diabetic retinopathy prediction being within a predetermined range.

8. The non-transitory computer readable medium of illustrative embodiment 7, wherein the at least five patient predictors are in a range from five patient predictors to ten patient predictors.

9. The non-transitory computer readable medium of any of illustrative embodiments 7 to 8, further comprising computer executable instructions that when executed cause the processor to display a user interface on a display, the user interface having a plurality of fields operable to receive input from a user, the input indicative of the at least five patient predictors.

10. The non-transitory computer readable medium of illustrative embodiment 9, wherein the user is at least one of a patient and a physician.

11. The non-transitory computer readable medium of any of illustrative embodiments 7 to 10, further comprising computer executable instructions that when executed cause the processor to pass an alert to a user responsive to the diabetic retinopathy prediction being within a predetermined range.

12. The non-transitory computer readable medium of illustrative embodiment 11, wherein the user is at least one of a patient and a physician.

13. The non-transitory computer readable medium of illustrative embodiment 11, wherein the alert is provided in at least one of an aural form or a visual form.

14. The non-transitory computer readable medium of illustrative embodiment 11, wherein the alert is indicative of at least one of an eye examination recommendation and an eye examination frequency.

15. A non-transitory computer readable medium having computer executable instructions that when executed cause a processor to:
determine, by a first algorithm, a diabetic retinopathy prediction based at least in part on a weighted sum of at least five patient predictors in a database storing at least one patient record having first data; and
pass data indicative of the diabetic retinopathy prediction to a second algorithm responsive to the diabetic retinopathy prediction being within a predetermined range.

16. The non-transitory computer readable medium of illustrative embodiment 15, wherein the second algorithm is operable to generate a readmission prediction.

17. The non-transitory computer readable medium of any of illustrative embodiments 15 to 16, wherein the at least five patient predictors are in a range from five patient predictors to ten patient predictors.

18. The non-transitory computer readable medium of any of illustrative embodiments 15 to 17, further comprising computer executable instructions that when executed cause the processor to display a user interface on a display, the user interface having a plurality of fields operable to receive input from a user, the input indicative of the at least five patient predictors.

19. The non-transitory computer readable medium of illustrative embodiment 18, wherein the user is at least one of a patient and a physician.

20. The non-transitory computer readable medium of illustrative embodiment 16, further comprising computer executable instructions that when executed cause the processor to pass an alert to a user responsive to the readmission prediction being within a predetermined range.

### CONCLUSION

DR is a major cause of blindness among middle-aged adults over the world. Vision loss which occurs at the late stage of DR cannot be reversed. As a result, diagnosing DR at an early date is very desirable. The present disclosure describes a system for calculating the risk of developing DR for a patient with diabetes and a method of using the same in a practical application. The system and method may apply a risk index to a number of patient predictors for DR, which may include the patient's age, status on neuropathy and nephropathy, and results for analyzing multiple analytes, e.g., eight analytes (i.e., creatinine, HbA1c, white blood cell, glucose, hematocrit, anion gap, potassium, and sodium) along with other patient data. Using the patient predictors as inputs, a user may employ the presently described system and method to evaluate the patient's risk of developing DR and perform an action such as alerting the user to see an ophthalmologist for eye examination and potentially follow-up actions recommended by the ophthalmologist.

One technological improvement of the presently described system and method is the determination of a subset of patient predictors 18 that contribute a majority of predictive accuracy and development of the first algorithm that improves the functioning of the processor by reducing clock cycles as compared to existing methodologies using machine learning techniques. As a result, data collection may be less expensive and collected data may be easier to interpret. Additionally, while existing methods for predicting DR utilize machine learning and may achieve high predictive accuracy, the machine learning algorithms require significant processing power and memory, and the “black box” nature of such methods make them difficult for a user to understand. Moreover, complex machine learning algorithms require the support of specific software (e.g., R) for their execution, which may be less user-friendly and may increase the cost of use. In order to address these concerns, another improvement of the presently described system and method is the development of a risk index and the optional inclusion of a graphical user interface. The DR prediction given in accordance with the present disclosure may be used as an early warning sign to urge patients to undergo an ophthalmic examination, which has a relatively low compliance rate currently.

From the above description, it is clear that the inventive concepts disclosed herein is well adapted to carry out the objects and to attain the advantages mentioned herein as well as those inherent in the inventive concepts disclosed herein. While presently preferred embodiments of the inventive concepts disclosed herein have been described for purposes of this disclosure, it will be understood that numerous changes may be made which will readily suggest themselves to those skilled in the art and which are accomplished within the scope and coverage of the inventive concepts disclosed and claimed herein.

What is claimed is:

1. A non-transitory computer readable medium having computer executable instructions that when executed cause a processor to:
determine a diabetic retinopathy prediction based at least in part on a weighted sum of at least five patient predictors in a database storing at least one patient record having first data; and
pass an alert to a user responsive to the diabetic retinopathy prediction being within a predetermined range.

2. The non-transitory computer readable medium of claim 1, wherein the user is at least one of a patient and a physician.

3. The non-transitory computer readable medium of claim 1, wherein the alert is provided in at least one of an aural form or a visual form.

4. The non-transitory computer readable medium of claim 1, wherein the alert is indicative of at least one of an eye examination recommendation and an eye examination frequency.

5. The non-transitory computer readable medium of claim 1, wherein the at least five patient predictors are in a range from five patient predictors to ten patient predictors.

6. The non-transitory computer readable medium of claim 1, further comprising computer executable instructions that when executed cause the processor to display a user interface on a display, the user interface having a plurality of fields operable to receive input from a user, the input indicative of the at least five patient predictors.

7. A non-transitory computer readable medium having computer executable instructions that when executed cause a processor to:
determine a diabetic retinopathy prediction based at least in part on a weighted sum of at least five patient predictors in a database storing at least one patient record having first data; and
update the first data with second data responsive to the diabetic retinopathy prediction being within a predetermined range.

8. The non-transitory computer readable medium of claim 7, wherein the at least five patient predictors are in a range from five patient predictors to ten patient predictors.

9. The non-transitory computer readable medium of claim 7, further comprising computer executable instructions that when executed cause the processor to display a user interface on a display, the user interface having a plurality of fields operable to receive input from a user, the input indicative of the at least five patient predictors.

10. The non-transitory computer readable medium of claim 9, wherein the user is at least one of a patient and a physician.

11. The non-transitory computer readable medium of claim 7, further comprising computer executable instructions that when executed cause the processor to pass an alert to a user responsive to the diabetic retinopathy prediction being within a predetermined range.

12. The non-transitory computer readable medium of claim 11, wherein the user is at least one of a patient and a physician.

13. The non-transitory computer readable medium of claim 11, wherein the alert is provided in at least one of an aural form or a visual form.

14. The non-transitory computer readable medium of claim 11, wherein the alert is indicative of at least one of an eye examination recommendation and an eye examination frequency.

15. A non-transitory computer readable medium having computer executable instructions that when executed cause a processor to:
determine, by a first algorithm, a diabetic retinopathy prediction based at least in part on a weighted sum of at least five patient predictors in a database storing at least one patient record having first data; and
pass data indicative of the diabetic retinopathy prediction to a second algorithm responsive to the diabetic retinopathy prediction being within a predetermined range.

16. The non-transitory computer readable medium of claim 15, wherein the second algorithm is operable to generate a readmission prediction.

17. The non-transitory computer readable medium of claim 15, wherein the at least five patient predictors are in a range from five patient predictors to ten patient predictors.

18. The non-transitory computer readable medium of claim 15, further comprising computer executable instructions that when executed cause the processor to display a user interface on a display, the user interface having a plurality of fields operable to receive input from a user, the input indicative of the at least five patient predictors.

19. The non-transitory computer readable medium of claim 18, wherein the user is at least one of a patient and a physician.

20. The non-transitory computer readable medium of claim 16, further comprising computer executable instructions that when executed cause the processor to pass an alert to a user responsive to the readmission prediction being within a predetermined range.
